(19) United States
(12) Patent Application Publication
(10) Pub. No.: US 2025/0268576 A1
(43) Pub. Date: Aug. 28, 2025

(54) ULTRASOUND DIAGNOSTIC APPARATUS, METHOD, AND STORAGE MEDIUM

(71) Applicant: CANON MEDICAL SYSTEMS CORPORATION, Tochigi (JP)

(72) Inventors: Daiki ITO, Utsunomiya (JP); Masaki WATANABE, Utsunomiya (JP); Hiroki YOSHIARA, Utsunomiya (JP); Yu IGARASHI, Utsunomiya (JP)

(73) Assignee: CANON MEDICAL SYSTEMS CORPORATION, Tochigi (JP)

(21) Appl. No.: 19/065,504

(22) Filed: Feb. 27, 2025

(30) Foreign Application Priority Data
Feb. 27, 2024 (JP) 2024-027458

(51) Int. Cl. A61B 8/00; G16H 50/30

(52) U.S. Cl. CPC A61B 8/5223; A61B 8/461; A61B 8/485; G16H 50/30

(57) ABSTRACT

An ultrasound diagnostic apparatus of an embodiment includes processing circuitry. The processing circuitry is configured to measure a plurality of tissue property parameters based on a reflected wave signal received from a subject. The processing circuitry is configured to calculate a reliability index of an index value based on the plurality of tissue property parameters, based on each of reliabilities of the plurality of tissue property parameters.

### CROSS-REFERENCE TO RELATED APPLICATIONS

This application is based upon and claims the benefit of priority from Japanese Patent Application No. 2024-027458, filed on Feb. 27, 2024; the entire contents of which are incorporated herein by reference.

### FIELD

Embodiments described herein relate generally to an ultrasound diagnostic apparatus, a method, and a storage medium.

### BACKGROUND

An ultrasound diagnostic apparatus has a function of visualizing morphology of living tissue based on reflected wave signals of ultrasonic waves transmitted from an ultrasound probe, in addition to a function of quantifying tissue properties. For example, the ultrasound diagnostic apparatus can measure tissue property parameters, such as elasticity and viscosity of living tissue, by measuring a propagation speed of shear waves generated by a push pulse. Moreover, the ultrasound diagnostic apparatus can measure tissue property parameters indicating the attenuation of ultrasonic waves in a living tissue by analyzing the attenuation state of the reflected wave signals. Such tissue property parameters are used as index values for determining severity of, for example, liver fibrosis, hepatitis, fatty liver, and the like.

Moreover, in recent years, a method for non-invasively diagnosing non-alcoholic steatohepatitis (NASH) by statistical analysis, machine learning, and the like using the tissue property parameters described above have been considered as an alternative to liver biopsy. For example, a diagnostic method based on index values using multiple tissue property parameters have been considered.

### BRIEF DESCRIPTION OF THE DRAWINGS

FIG. 1 is a block diagram illustrating an example of a configuration of an ultrasound diagnostic apparatus according to a first embodiment;

FIG. 2 is a flowchart illustrating a procedure of processing of the ultrasound diagnostic apparatus according to the first embodiment;

FIG. 3A is a diagram illustrating an example of a result of measurement processing according to the first embodiment;

FIG. 3B is a diagram illustrating an example of a result of measurement processing according to the first embodiment;

FIG. 3C is a diagram illustrating an example of a result of measurement processing according to the first embodiment;

FIG. 4A is a diagram for explaining an example of acquisition a reliability according to the first embodiment;

FIG. 4B is a diagram for explaining an example of acquisition a reliability according to the first embodiment;

FIG. 5 is a diagram for explaining an example of extraction of a high reliability area according to the first embodiment;

FIG. 6 is a diagram illustrating an example of display information according to the first embodiment; and

FIG. 7 is a diagram illustrating an example of the display information according to the first embodiment.

### DETAILED DESCRIPTION

An ultrasound diagnostic apparatus according to an embodiment includes processing circuitry. The processing circuitry is configured to measure a plurality of tissue property parameters based on a reflected wave signal received from a subject. The processing circuitry is configured to calculate a reliability index of an index value based on the plurality of tissue property parameters, based on each of reliabilities of the plurality of tissue property parameters.

Hereinafter, embodiments of an ultrasound diagnostic apparatus, a method, and a computer program according to the present application will be explained in detail with reference to the accompanying drawings. The ultrasound diagnostic apparatus, the method, and the computer program according to the present application are not limited to the embodiments described below.

### First Embodiment

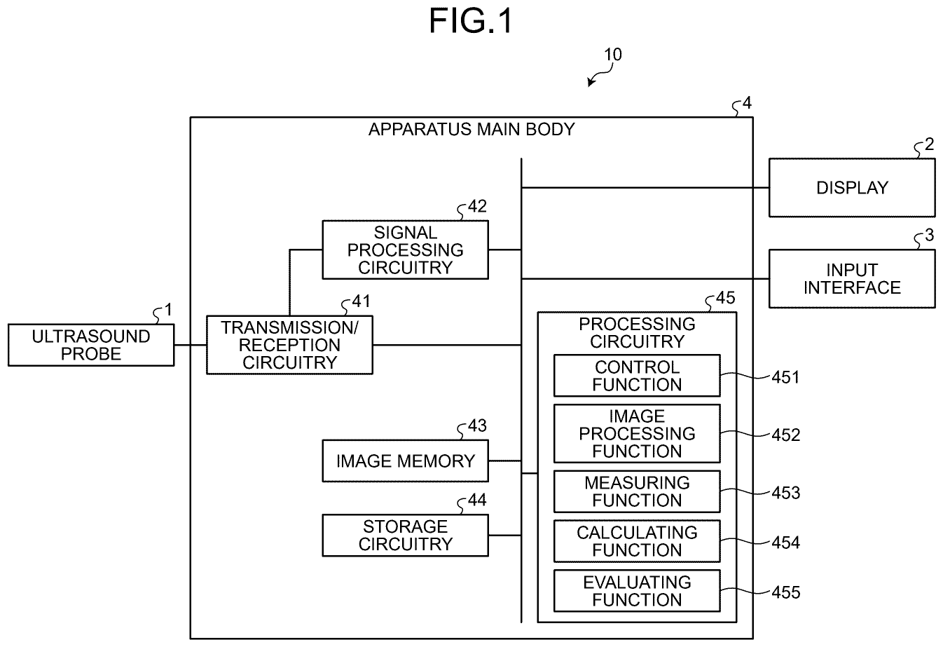

FIG. 1 is a block diagram illustrating an example of a configuration of an ultrasound diagnostic apparatus 10 according to the first embodiment. As illustrated in FIG. 1, the ultrasound diagnostic apparatus 10 according to the present embodiment includes an ultrasound probe 1, a display 2, an input interface 3, and an apparatus main body 4, and the ultrasound probe 1, the display 2, and the input interface 3 are connected in a communication enabled manner with the apparatus main body 4.

The ultrasound probe 1 has multiple piezoelectric transducers, and these piezoelectric transducers generate ultrasonic waves based on a driving signal provided by transmission reception circuitry 41. Moreover, the ultrasound probe 1 receives a reflected wave from a subject, to convert into an electrical signal. Furthermore, the ultrasound probe 1 includes a matching layer provided on the piezoelectric transducer, a backing material that prevents propagation of ultrasonic waves toward the rear from the piezoelectric transducer, and the like. The ultrasound probe 1 is detachably connected to the apparatus main body 4.

When ultrasonic waves are transmitted to the subject from the ultrasound probe 1, the transmitted ultrasonic waves are reflected successively on a discontinuous surface of an acoustic impedance in a body tissue of the subject, and are received by the piezoelectric transducers included in the ultrasound probe 1 as reflected wave signals. An amplitude of a received reflected wave signal is dependent on a difference in the acoustic impedance on the discontinuous surface on which the ultrasonic wave is reflected. A reflected wave signal obtained when a transmitted ultrasonic wave pulse is reflected on a moving blood flow, a surface of a heart wall, or the like is subject to frequency shift depending on a velocity component with respect to a direction of transmission of an ultrasonic wave of a moving body due to the Doppler effect.

The ultrasound probe 1 may be a one-dimensional ultrasound probe in which multiple piezoelectric transducers are arranged in a single row, or may be an ultrasound probe that mechanically oscillates multiple piezoelectric transducers of a one-dimensional ultrasound probe, or a two-dimensional ultrasound probe in which multiple piezoelectric transducers are arranged in a grid pattern in two dimensions.

The display 2 displays a graphical user interface (GUI) for an operator of the ultrasound diagnostic apparatus 10 to input various kinds of setting requests using the input interface 3, an ultrasound image generated in the apparatus main body 4, and the like. Moreover, the display 2 displays various kinds of messages and display information to notify the operator of a processing status and a processing result of the apparatus main body 4. Furthermore, the display 2 includes a speaker, and is capable of outputting sound.

The input interface 3 is operated to perform settings for a predetermined position (for example, the position of a region of interest (ROI)) and the like, and is implemented by, for example, a trackball, a switch button, a mouse, a keyboard, a touchpad for performing input operations by touching an operation surface, a touch monitor integrating a display screen and a touchpad, a non-contact input circuit using an optical sensor, a sound input circuit, and the like. The input interface 3 is connected to processing circuitry 45 described later, and converts an input operation accepted from the operator into an electrical signal, to output to the processing circuitry 45. In the present embodiment, the input interface 3 is not limited to be configured to include physical operation components, such as a mouse and a keyboard. For example, examples of the input interface include an electric signal processing circuit that receives an electrical signal corresponding to an input operation from an external input device provided separately from the apparatus and that outputs the electric signal to the processing circuitry 45.

The apparatus main body 4 is an apparatus to generate an ultrasound image data based on a reflected wave signal received by the ultrasound probe 1, and includes the transmission/reception circuitry 41, signal processing circuitry 42, an image memory 43, storage circuitry 44, and the processing circuitry 45 as illustrated in FIG. 1. The transmission/reception circuitry 41, the signal processing circuitry 42, the image memory 43, the storage circuitry 44, and the processing circuitry 45 are connected to one another in a communication enabled manner. In the ultrasound diagnostic apparatus 10 illustrated in FIG. 1, respective processing functions are stored in the storage circuitry 44 in a form of computer-executable program. The transmission/reception circuitry 41, the signal processing circuitry 42, and the processing circuitry 45 are processors that implement a function corresponding to the respective programs by reading and executing a program from the storage circuitry 44. In other words, the respective circuitry that have read the respective programs are to have a function corresponding to the read program.

The transmission/reception circuitry 41 includes a pulse generator, a transmission delay unit, a pulser, and the like, and provides a driving signal to the ultrasound probe 1. The pulse generator repeatedly generates rate pulses at a predetermined rate frequency to form a transmission ultrasonic wave. The transmission delay unit applies to each rate pulse generated by the pulse generator a delay for each piezoelectric transducer necessary to focus ultrasonic waves generated by the ultrasound probe 1 into a beam shape, and to determine a transmission directivity. The pulser applies a driving signal (driving pulse) to the ultrasound probe 1 at a timing based on the rate pulse. That is, the transmission delay unit varies the delay to be applied to each rate pulse, and thereby adjusts the transmission direction of an ultrasonic wave to be transmitted from a piezoelectric transducer surface arbitrarily.

The transmission/reception circuitry 41 has a function of instantly changing a transmission frequency, a transmission drive voltage, and the like to perform a predetermined scan sequence based on an instruction of the processing circuitry 45 describe later. Particularly, changing the transmission drive voltage is achieved by a linear-amplifier oscillation circuit capable of instantly switching its value, or by a mechanism that electrically switches between multiple power supply units.

Furthermore, the transmission/reception circuitry 41 includes a preamplifier, an analog-to-digital (A/D) converter, a reception delay unit, an adder, and the like, and generates reflected wave data by performing various kinds of processing with respect to a reflected wave signal received by the ultrasound probe 1. The preamplifier amplifies the reflected wave signal per channel. The A/D converter A/D converts the amplified reflected wave signal. The reception delay unit applies a delay necessary to determine a reception directivity. The adder performs addition processing of the reflected waved signals processed by the reception delay unit, to generate reflected wave data. By the addition processing of the adder, a reflection component from a direction corresponding to the reception directivity of the reflected wave signal is emphasized, and a comprehensive beam of ultrasound transmission and reception is formed based on the reception directivity and transmission directivity.

The signal processing circuitry 42 performs logarithmic amplification, envelope detection processing, and the like on the reflected wave signal received from the transmission/reception circuitry 41, to generate data (B-mode data) representing signal strength of each sample point with brightness. The B-mode data generated by the signal processing circuitry 42 is output to the processing circuitry 45.

Moreover, the signal processing circuitry 42 generates data (Doppler data) obtained by extracting movement information based on the Doppler effect of a moving object at each sample point within a scanning region from the reflected signal data received from the transmission/reception circuitry 41. Specifically, the signal processing circuitry 42 performs frequency analysis on velocity information from the reflected wave data, extracts blood flow, tissue, and contrast agent echo components based on the Doppler effect, and generates data (Doppler data) obtained by extracting moving object information, such as mean velocity, dispersion, and power, at multiple points. The moving object is, for example, blood flow, a tissue of a heart wall or the like, and a contrast agent. The movement information (blood flow information) acquired by the signal processing circuitry 42 is transmitted to the processing circuitry 45, and displayed in color on the display 2 as a mean velocity image, a dispersion image, a power image, or a combination image of these.

The image memory 43 is a memory that stores image data for display generated by the processing circuitry 45. Moreover, the image memory 43 can store data generated by the signal processing circuitry 42. The B-mode data and the Doppler data stored in the image memory 43 can be retrieved by an operator after diagnosis, and is to be an ultrasound image for display via the processing circuitry 45.

The storage circuitry 44 stores a control program to perform ultrasound transmission/reception, image processing, and display processing, and various kinds of data such as diagnosis information (for example, patient ID, findings of a doctor, and the like, a diagnostic protocol, and various body marks. Furthermore, the storage circuitry 44 stores processing results of the transmission/reception circuitry 41, the signal processing circuitry 42, and the processing circuitry 45. Moreover, the storage circuitry 44 is also used for storing image data that is stored in the image memory 43 as necessary. Furthermore, the data stored by the storage circuitry 44 can be transferred to an external device through an interface not illustrated.

The processing circuitry 45 controls overall processing of the ultrasound diagnostic apparatus 10. Specifically, the processing circuitry 45 controls processing of the transmission/reception circuitry 41 and the signal processing circuitry 42 based on various kinds of setting request input by the operator through the input interface 3, or various kinds of control programs and various kinds of data read from the storage circuitry 44. Moreover, the processing circuitry 45 controls to display the ultrasound image for display stored in the image memory 43 on the display 2.

The processing circuitry 45 performs, as illustrated in FIG. 1, a control function 451, an image processing function 452, a measuring function 453, a calculating function 454, and an evaluating function 455. The control function 451 is one example of a display control unit. The measuring function 453 is one example of a measuring unit. The calculating function 454 is one example of a calculating unit. The evaluating function is one example of an evaluating unit.

The control function 451 controls processing of the transmission/reception circuitry 41 and the signal processing circuitry 42 based on various kinds of setting request input by the operator through the input interface 3, or various kinds of control programs and various kinds of data read from the storage circuitry 44. The control function can control transmission and reception of ultrasonic waves to measure a tissue property parameter including elasticity and viscosity of a tissue of the subject, attenuation of an ultrasonic wave, and the like. Moreover, the control function 451 controls to display an ultrasound image or various display information on the display 2. For example, the control function 451 displays an index value and a reliability index based on multiple tissue property parameters on the display 2. The display by the control function 451 will be described in detail later.

The image processing function 452 generates an ultrasound image from data generated by the signal processing circuitry 42. That is, the image processing function 452 generates an ultrasound image representing a strength of a reflected wave using brightness from the B-mode data generated by the signal processing circuitry 42. Furthermore, the image processing function 452 generates an ultrasound image representing moving object information (blood flow information or movement information of a tissue) from the Doppler data generated by the signal processing circuitry 42. The ultrasound image based on the Doppler data includes velocity image data, dispersion image data, power image data, and combined image data of these.

Generally, the image processing function 452 converts (scan converts) a scan line signal sequence of ultrasound scanning into a scan line signal sequence of a video format represented by television and the like, to generate an ultrasound image for display. Specifically, the image processing function 452 generates an ultrasound image for display by performing coordinate conversion according to a scanning mode of ultrasonic waves by the ultrasound probe 1. Moreover, besides the scan conversion, the image processing function 452 performs, for example, image processing (smoothing processing) to regenerate a mean luminance image by using plural image frames subjected to the scan conversion, image processing (edge enhancement processing) using a differential filter in an image, and the like as various kinds of image processing. Furthermore, the image processing function 452 superimposes character information of various parameters, scales, body marks, and the like on the ultrasound image data.

That is, the B-mode data and the Doppler data are ultrasound image data before scan conversion processing, and data generated by the image processing function 452 is ultrasound image data for display after scan conversion processing. The image processing function 452 generates, when the signal processing circuitry 42 has generated three-dimensional data (three-dimensional B-mode data and three-dimensional Doppler data), generates volume data by performing coordinate conversion according to a scanning mode of ultrasonic wave by the ultrasound probe 1. The image processing function 452 then performs various kinds of rendering processing with respect to the volume data, to generate two-dimensional image data for display.

The measuring function 453 measures multiple tissue property parameters based on a reflected wave signal received from a subject. Specifically, the measuring function 453 measures tissue property parameters including at least two of elasticity of a tissue, viscosity of a tissue, and attenuation of an ultrasonic wave. For example, the measuring function 453 measures a parameter indicating elasticity of a tissue by calculating a propagation speed of a shear wave based on a reflected wave signal. The measuring function 453 can also calculate an elasticity modulus (Young's modulus, shear elasticity modulus) from a propagation speed of a shear wave.

Moreover, the measuring function 453 measures a parameter indicating viscosity of a tissue, for example, based on a relationship between a frequency of a shear wave and a propagation speed of the shear wave. As one example, the measuring function 453 calculates a slope of phase velocity distribution as a parameter indicating the viscosity of a tissue. The measuring function 453 can also calculate a viscosity modulus.

Moreover, the measuring function 453 measures a parameter indicating attenuation of an ultrasonic wave, for example, based on a reflected wave signal. As one example, the measuring function 453 calculates an attenuation amount of a transmitted ultrasonic wave as a parameter indicating the attenuation of an ultrasonic wave.

Moreover, the measuring function 453 calculates an index value based on multiple tissue property parameters, based on multiple tissue property parameters and a regression model or a machine learning model based on statistical analysis. For example, the measuring function 453 calculates the index value using the elasticity of a tissue, the viscosity of a tissue, and a result of attenuation of an ultrasonic wave. The processing performed by the measuring function 453 will be described in detail later.

The calculating function 454 calculates a reliability index of an index value based on multiple tissue property parameters, based on reliabilities of respective tissue property parameters. Specifically, the calculating function 454 acquires reliabilities of the respective tissue property parameters, and calculates a reliability index that indicates the reliability of the index value calculated by the measuring function 453 based on the acquired reliabilities. The processing performed by the calculating function 454 will be described in detail later.

The evaluating function 455 evaluates the index value based on multiple tissue property parameters, based on the reliability index. Specifically, the evaluating function 455 evaluates an index value calculated by the measuring function 453 based on the reliability index calculated by the calculating function 454. The processing performed by the evaluating function 455 will be described in detail later.

The ultrasound diagnostic apparatus 10 according to the first embodiment enables to appropriately perform analysis of an index value. For example, in an analysis using a tissue property parameter, a diagnostic method using an index value based on multiple tissue property parameters is considered, but it is difficult to determine whether data corresponding to a region in which the index value is analyzed is reliable. As a result, subjectivity of an operator can be involved, and it can sometimes prevent a proper analysis of the index value.

Accordingly, the ultrasound diagnostic apparatus 10 calculates a reliability index of an index value based on multiple tissue property parameters based on the reliability of each of the multiple tissue property parameters, to thereby enable to perform the analysis of the index value appropriately.

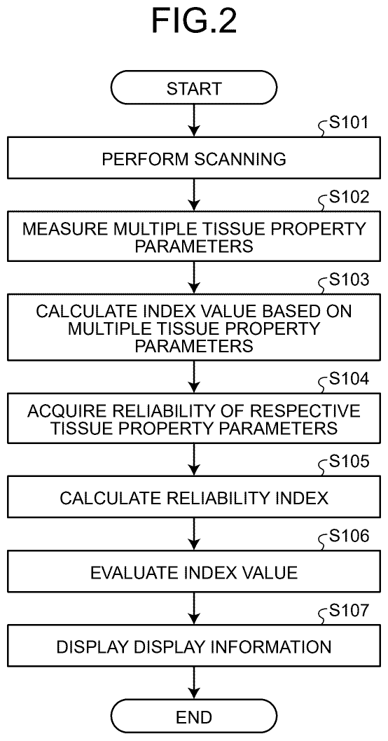

Hereinafter, a procedure of processing performed by the ultrasound diagnostic apparatus 10 will be explained using FIG. 2, and then details of respective processing will be explained. FIG. 2 is a flowchart illustrating the procedure of processing performed by the ultrasound diagnostic apparatus according to the first embodiment.

For example, as illustrated in FIG. 2, in the present embodiment, the control function 451 performs scanning to measure a tissue property parameter (step S101). For example, the control function 451 performs scanning to perform measurement by shear wave elastography (SWE), shear wave dispersion (SWD), and attenuation imaging (ATI). The processing at step S101 described above is achieved, for example, by the processing circuitry 45 retrieving a program corresponding to the control function 451 from the storage circuitry 44, and executing it.

Subsequently, the measuring function 453 measures multiple tissue property parameters based on data acquired by the scanning (step S102). Furthermore, the measuring function 453 calculates an index value based on the multiple tissue property parameters based on a measurement result (step S103). For example, the measuring function 453 measures elasticity of a tissue, viscosity of a tissue, and attenuation of an ultrasonic wave, and calculates an index value based on these results. The processing at steps S102 and S103 described above is achieved, for example, by the processing circuitry 45 retrieving a program corresponding to the measuring function 453 from the storage circuitry 44, and executing it.

Subsequently, the calculating function 454 acquires reliabilities of the respective tissue property parameters (step S104), and calculates a reliability index indicating the reliability of an index value based on the acquired reliabilities of the respective tissue property parameters (step S105). The processing at steps S104 and S105 described above is achieved, for example, by the processing circuitry 45 retrieving a program corresponding to the calculating function 454 from the storage circuitry 44, and executing it.

Subsequently, the evaluating function 455 evaluates the index value based on the calculated reliability index (step S106). The processing at step S106 described above is achieved, for example, by the processing circuitry 45 retrieving a program corresponding to the evaluating function 455 from the storage circuitry 44, and executing it.

Subsequently, the control function 451 controls to display information (reliability index, index value, and the like) on the display 2 (step S107). The processing at step S107 described above are achieved, for example, by the processing circuitry 45 retrieving a program corresponding to the control function 451 from the storage circuitry 44, and executing it.

Hereinafter, details of the respective processing performed by the ultrasound diagnostic apparatus 10 will be explained.

### Scanning

As explained at step S101, the control function 451 performs scanning to measure multiple tissue property parameters. For example, the control function 451 transmits an ultrasonic wave (push pulse) for displacement generation for generating displacement to induce a shear wave in a subject, and controls to transmit and receive an ultrasonic wave (tracking pulse) for displacement observation for observing the generated shear wave when measurement by SWE and SWD is to be performed.

For example, the control function 451 causes the ultrasound probe 1 to transmit a push pulse, to generate a shear wave in a living tissue. The control function 451 then causes the ultrasound probe 1 to transmit a tracking pulse for observing the shear wave that generates based on the push pulse. The tracking pulse is transmitted to observe a propagation speed of a shear wave generated by the push pulse at each sample point within a measurement area. Normally, tracking pulses are transmitted multiple times (for example, 100 times) for each scan line within the measurement area. The control function 451 generates reflected wave data (scan data) from a reflected wave signals of the tracking pulses transmitted along each scan line within the measurement area.

Furthermore, for example, when measurement by ATI is to be performed, the control function 451 performs scanning under conditions similar to those of scanning in a standard B-mode scanning. ATI utilizes attenuation of an ultrasonic wave transmitted into a living body caused as it passes through a tissue due to absorption, scattering, and the like, and it can be measured from scan data acquired in B-mode.

### Measurement Processing of Tissue Property Parameter

As explained at step S102, the measuring function 453 measures multiple tissue property parameters based on scan data generated by the control performed by the control function 451. For example, when elasticity of a tissue is to be measured by SWE, the measuring function 453 acquires time variation information of displacement caused by the shear wave for each position in a region of interest (ROI) corresponding to a scan range from scan data generated from the reflected wave signal of the tracking pulse, determines an arrival time of the shear wave for each position in the region of interest based on the acquired time variation information of displacement, and determines a speed of the shear wave based on the determined arrival time.

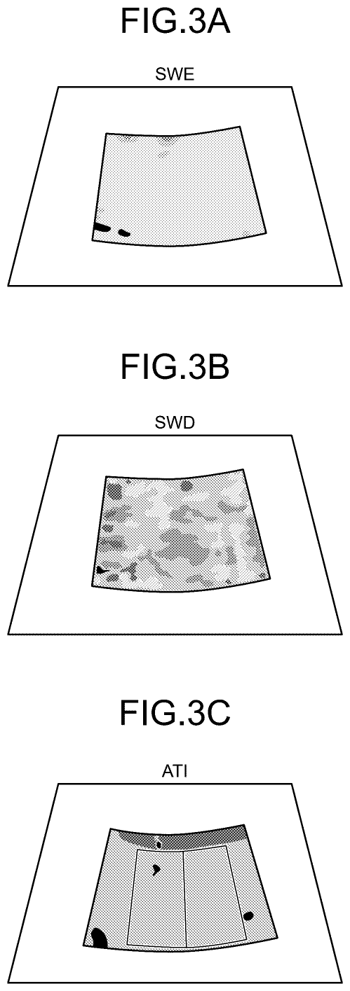

The image processing function 452 can generate an elasticity image (image expressing elasticity) illustrated in FIG. 3A by allocating a pixel value according to the speed of the shear wave determined as described above to each position in the region of interest. FIG. 3A is a diagram illustrating an example of a result of the measurement processing according to the first embodiment.

Moreover, for example, when viscosity of a tissue is to be measured by SWD, the measuring function 453 acquires time variation information of displacement caused by the shear wave for each position in a region of interest from scan data generated from the reflected wave signal of the tracking pulse, performs frequency analysis of the acquired time variation information of displacement, to generate a distribution indicating a relationship between a shear wave and a frequency for each position in the region of interest, and determines a viscosity value based on the relationship.

The image processing function 452 can generate viscosity image (image expressing viscosity) illustrated in FIG. 3B by allocating a viscosity value determined as described above to each position in the region of interest. FIG. 3B is a diagram illustrating an example of a result of the measurement processing according to the first embodiment.

Moreover, for example, when attenuation is to be measured by ATI, the measuring function 453 acquires processed scan data by performing to offset a signal amplification due to various gains, and processing to counteract an effect of an acoustic field with respect to the scan data acquired in B-mode, and determines an attenuation index value (attenuation coefficient) for each position in the region of interest by differentiating the acquired processed scan data along a transmission/reception direction (depth direction) of an ultrasonic wave.

The image processing function 452 can generate attenuation image (image expressing attenuation of an ultrasonic wave) illustrated in FIG. 3C by allocating an attenuation index value determined as described above to each position in the region of interest. FIG. 3C is a diagram illustrating an example of a result of the measurement processing according to the first embodiment.

### Calculation Processing of Index Value

As explained at step S103, the measuring function 453 calculates an index value based on the measured multiple tissue property parameters. For example, the measuring function 453 calculates an index value for evaluating a presence or a progression of a disease based on the result of SWE, the result of SWD, and the result of ATI.

As an example, the measuring function 453 calculates a score for evaluating a liver disease from the result of SWE, the result of SWD, and the result of ATI using a regression model or a machine learning model based on statistical analysis for each pixel. The regression model described above is a model calculated by logistic regression, and the machine learning model is a model acquired by, for example, support vector machine or a random forest. These models are established in advance such that a score expressing presence or absence, or a progression of a liver disease, such as non-alcoholic steatohepatitis (NASH), can be obtained, for example, from the result of SWE, the result of SWD, and the result of ATI, and are stored in the storage circuitry 44. That is, the measuring function 453 calculates an index value in which multiple tissue property parameters are combined using these models read from the storage circuitry 44.

In the example described above, measurement of parameters by SWE, SWD, and ATI has been explained, but embodiments are not limited thereto. As long as it is a parameter expresses a tissue property, other parameters may be measured. Moreover, in the example described above, a case in which an index value in which SWE, SWD, and ATI are combined is calculated has been explained, but embodiments are not limited thereto. Other than that, an index value of arbitrary combination may be calculated. For example, an index value may be calculated by combination of arbitrary two out of SWE, SWD, and ATI, a combination of at least one of SWE, SWD, and ATI and another parameter, or a combination of other parameters.

### Acquisition Processing of Reliability

As explained at step S104, the calculating function 454 acquires respective reliabilities of multiple tissue property parameters measured by the measuring function 453. For example, the calculating function 454 acquires a reliability regarding elasticity and viscosity of a tissue based on at least one of amplitude of a shear wave, a signal-to-noise (S/N) ratio, a propagation accuracy of a shear wave, and a standard deviation of numerical values.

For example, the calculating function 454 acquires a reliability based on a relationship between a propagation speed of a shear wave and an amplitude of a shear wave. The propagation speed of a shear wave is slower in a soft tissue and faster in a hard tissue. Moreover, the amplitude of a shear wave is higher in a soft tissue, and lower in a hard tissue. The calculating function 454 quantifies a degree of consistency between the propagation speed and the amplitude of a shear wave using these relationships to acquire as a reliability for each position in the region of interest. Furthermore, the calculating function 454 can acquire an S/N ratio of the scan data as a reliability.

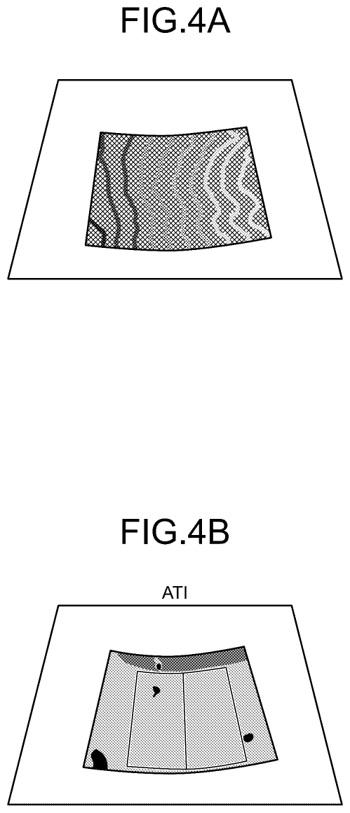

Moreover, the calculating function 454 can acquire a reliability from the propagation image (propagation map) indicating the accuracy of propagation of a shear wave. FIG. 4A is a diagram for explaining an example of acquisition of a reliability according to the first embodiment. FIG. 4A illustrates a propagation image. As illustrated in FIG. 4A, the propagation image is displayed as a linear image (line image) formed by connecting positions at which the shear wave arrival times are approximately the same (for example, positions with substantially the same arrival time) with a line.

For example, in an environment in which there is no displacement caused by body movement of a subject, no reflection or refraction of a shear wave, and the elasticity and viscosity of a living tissue can be accurately measured, shear waves propagate almost evenly from a transmission position of the push pulse. Therefore, a line indicating the arrival time will be nearly parallel to the transmission direction of the push pulse and forms a curve according to the elasticity and viscosity of the living tissue. On the other hand, in an environment in which the elasticity and the viscosity of the living tissue cannot be accurately measured, the propagation of a shear wave may be observed as either extremely fast or extremely slow. Therefore, the line indicating the arrival time curves significantly. The calculating function 454 quantifies a reliability based on this propagation image, and acquires it for each position in the region of interest.

Furthermore, the calculating function 454 can acquire a reliability based on a standard deviation of values for each measured tissue property parameter. For example, the calculating function 454 can acquire a reliability by using a MAD (Measurement Area Detection) function of automatically detecting an area with a small standard deviation of value and calculating an average value. That is, the calculating function 454 acquires a magnitude of the standard deviation calculated by MAD as a reliability.

In the example described above, a case in which the amplitude of a shear wave, the S/N ratio, the propagation accuracy of a shear wave, and the standard deviation of values are used as a reliability regarding the elasticity and the viscosity of a tissue has been explained, but embodiments are not limited thereto. Any index that can be used as a reliability regarding elasticity and viscosity of a tissue may be used.

Moreover, the calculating function 454 acquires a reliability regarding attenuation of an ultrasonic wave based on at least one of accuracy of linear approximation of the reflected wave signal, multiple signals, and a structure. For example, the calculating function 454 acquires fitting accuracy (determination coefficient) when performing linear fitting on a change rate (slope) obtained from the signal intensity distribution of the processed scan data as a reliability.

Furthermore, the calculating function 454 can acquire a reliability, for example, by using a function of removing multiple reflections originating from an abdominal wall. FIG. 4B is a diagram for explaining an example of acquisition of a reliability according to the first embodiment. As illustrated in FIG. 4B, in B-mode, a region of interest can include an area (multiple reflection region) in which multiple reflections caused by the abdominal wall occur in a shallow part, and the function of removing multiple reflections extracts this multiple reflection region and removes it from the measurement result. The calculating function 454 acquires the extracted multiple reflection area as a low reliability area using this function.

Moreover, the calculating function 454 can acquire a reliability using a function of removing a structure in a region of interest. For example, as illustrated in a black-shaded area in FIG. 4B, when an area of a structure, such as a blood vessel, (structure area) is included in the region of interest, it is difficult to calculate an attenuation coefficient properly. Therefore, the function of removing a structure extracts this structure area, and removes it from the measurement result. The calculating function 454 acquires the extracted structure area as a low reliability area using this function.

In the example described above, a case in which accuracy of linear approximation of the reflected wave signal, multiple signals, and a structure are used as a reliability regarding attenuation of an ultrasonic wave has been explained, but embodiments are not limited thereto. Any index that can be used as a reliability regarding attenuation of an ultrasonic wave may be used.

### Calculation Processing of Reliability Index

As explained at step S105, the calculating function 454 calculates a reliability index of an index value based on multiple tissue property parameters using a reliability of each multiple tissue property parameter. Specifically, the calculating function 454 calculates a reliability index in which reliabilities acquired for the respective tissue property parameters are combined. For example, the calculating function 454 calculates a reliability index represented by a numerical value, a binary indicator, or a probability by combining numerical values of the acquired reliabilities for each position in a region of interest for the respective tissue property parameters (SWE, SWD, ATI). That is, the calculating function 454 calculates a reliability index for each pixel within the region of interest.

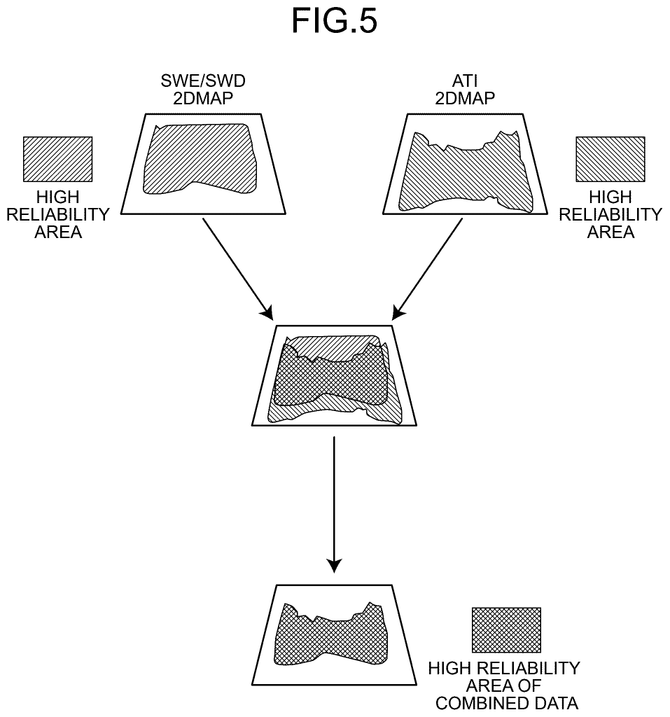

The calculating function 454 can extracts a high reliability area base on the reliability of the respective tissue property parameters. Specifically, the calculating function 454 can calculate a reliability index within an area in which a reliability of each of the multiple tissue property parameters is equal to or higher than a threshold in a tissue of a subject. FIG. 5 is a diagram for explaining an example of extraction of a high reliability area according to the first embodiment. For example, the calculating function 454 overlays a two-dimensional map (SWE/SWD 2DMAP) that indicates an area with high reliability in which the reliability in the measurement result by SWE and SWD exceeds a threshold with a two-dimensional map (ATI 2DMAP) that indicates an area with high reliability region in which the reliability in the measurement results by ATI exceeds a threshold as illustrated in FIG. 5, and extracts an overlapped area as the high reliability area.

The calculating function 454 can also extracts an area other than an area in which all of tissue property parameters exceed the threshold, as a high reliability area. For example, the calculating function 454 can calculate a reliability index within an area in which at least one of the reliabilities of the multiple tissue property parameters is equal to or higher than the threshold in a tissue of a subject. As one example, the calculating function 454 can extract an area illustrated in a middle part of FIG. 5 as a high reliability area.

### Evaluation Processing of Index Value

As explained at step S106, the evaluating function 455 evaluates the index value calculated by the measuring function 453 based on the reliability index calculated by the calculating function 454. For example, when the measuring function 453 calculates an index value (score) to diagnose presence or absence or progression of NASH by combining the measurement results of SWE, SWD, and ATI, the evaluating function 455 evaluates the index value based on the reliability index calculated by the calculating function 454.

As one example, the evaluating function 455 evaluates an index value of each position (each pixel) within a region of interest based on a threshold set for the reliability index calculated by the calculating function 454. That is, the evaluating function 455 evaluates a score calculated for each pixel in the region of interest based on the reliability index of a corresponding position. For example, the evaluating function 455 compares a reliability index with the threshold for each pixel within a high reliability area (area at a bottom in FIG. 5) extracted as an overlapped area in FIG. 5. The evaluating function 455 then evaluates which index value among the index values of respective pixels in the high reliability area have a reliability index that exceeds the threshold based on a comparison result. Thus, the evaluating function 455 can evaluate which index value is highly reliable, for example, among index values for diagnosing NASH.

### Display Processing of Display Information

As explained at step S107, the control function 451 controls to display, on the display 2, display information including the index value calculated by the measuring function 453 and the reliability index value calculated by the calculating function 454. For example, the control function 451 displays a two-dimensional color map indicating the reliability index for a region of interest in a tissue of a subject. That is, the control function 451 can display the two-dimensional color map in which a color according to a reliability index is allocated to each position within a scan range.

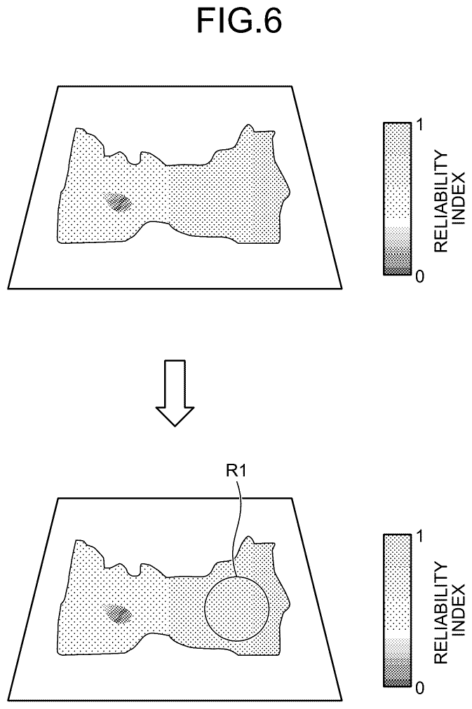

Furthermore, the control function 451 can a display two-dimensional color map indicating the reliability index only for a high reliability area. FIG. 6 is a diagram illustrating an example of the display information according to the first embodiment. For example, the control function 451 can display a two-dimensional color map in which a color according to a reliability index is allocated to a pixel within a high reliability area in which the reliabilities of all tissue property parameters exceed a threshold as illustrated in a drawing at an upper part of FIG. 6. That is, the control function 451 can present a high reliability area, and can further display a difference in reliability therein.

As described, by displaying the two-dimensional color map of a reliability index by the control function 451, an operator can grasp an area with a high reliability within a region of interest at a glance, and as illustrated in a drawing at a lower part of FIG. 6, a region, which is a measurement ROI to perform analysis of an index value can be set appropriately. Thus, the ultrasound diagnostic apparatus 10 enables to perform analysis of an index value appropriately regardless of which operator is operating.

The measuring function 453 can calculate various kinds of analysis values using an index value of a pixel included in the set measurement ROI. For example, the measuring function 453 calculates at least one of a mean value, a median value, and a standard deviation for a specified area (for example, region R1) based on an index value based on multiple tissue property parameters.

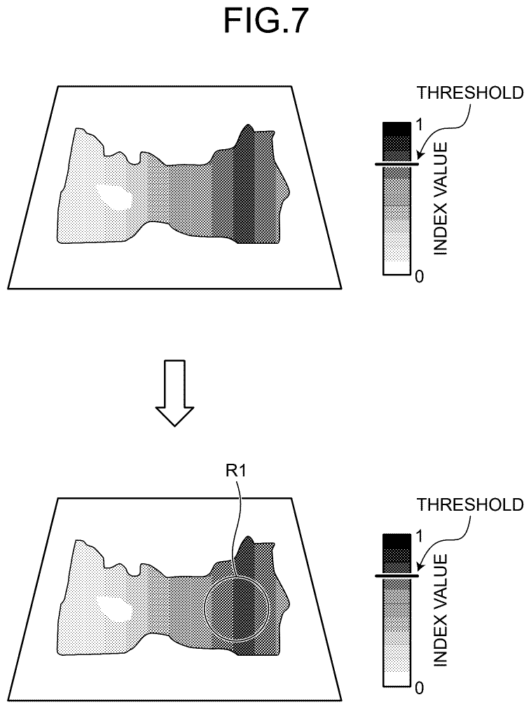

Moreover, the control function 451 can display an index value based on multiple tissue property parameters only for an area in which a reliability index is equal to or higher than a threshold in a tissue of a subject. FIG. 7 is a diagram illustrating an example of display information according to the first embodiment. For example, the control function 451 can display a two-dimensional color map in which a color according to an index value is allocated only to a pixel included in an area in which the reliability index exceeds a threshold within a high reliability area in which the reliabilities of all tissue property parameters exceed a threshold as illustrated in a drawing at an upper part of FIG. 7. That is, the control function 451 can display a state of the index value only for an area with a higher reliability within the high reliability area.

Thus, the operator can grasp an index value of an area with a higher reliability within the high reliability area at a glance, and can set the measurement ROI (region R1) appropriately as illustrated in a drawing illustrated in a lower part of FIG. 7.

The control function 451 can display a GUI for setting a threshold with respect to the index value as illustrated in FIG. 7. In such a case, the control function 451 can control to display only an index value that exceeds a set threshold (or value lower than the set threshold).

As described above, according to the first embodiment, the measuring function 453 measures multiple tissue property parameters based on a reflected wave signal received from a subject. The calculating function 454 calculates a reliability index of an index value based on the multiple tissue property parameters, based on each of the reliabilities of the respective multiple tissue property parameters. Therefore, the ultrasound diagnostic apparatus 10 according to the first embodiment can evaluate a reliability of the index value based on multiple tissue property parameters, and enables to perform analysis of the index value appropriately.

Moreover, according to the first embodiment, the multiple tissue property parameters include at least two of elasticity of a tissue, viscosity of a tissue, and attenuation of an ultrasonic wave. Therefore, the ultrasound diagnostic apparatus 10 according to the first embodiment enables to perform analysis of an index value regarding a liver disease appropriately.

Furthermore, according to the first embodiment, the calculating function 454 acquires a reliability regarding elasticity of a tissue and viscosity of a tissue based on at least one of amplitude of a shear wave, an S/N ratio, a propagation accuracy of a shear wave, and a standard deviation of numerical values. Therefore, the ultrasound diagnostic apparatus 10 according to the first embodiment can acquire an appropriate reliability regarding the elasticity and the viscosity of a tissue.

Moreover, according to the first embodiment, the calculating function 454 acquires a reliability regarding attenuation of an ultrasonic wave based on at least one of accuracy of linear approximation of the reflected wave signal, multiple signals, and a structure. Therefore, the ultrasound diagnostic apparatus 10 according to the first embodiment can acquire an appropriate reliability regarding the attenuation an ultrasonic wave.

Furthermore, according to the first embodiment, the calculating function 454 calculates a reliability index within an area in which respective reliabilities of multiple tissue property parameters are equal to or higher than a threshold in a tissue of a subject. Therefore, the ultrasound diagnostic apparatus 10 according to the first embodiment can acquire a reliability index of an index value for an area in which the reliabilities of the respective tissue property parameters are high.

Moreover, according to the first embodiment, the calculating function 454 calculates a reliability index within an area in which at least one of reliabilities of multiple tissue property parameters is equal to or higher than the threshold in a tissue of a subject. Therefore, the ultrasound diagnostic apparatus 10 according to the first embodiment can acquire a reliability index for an area in which the reliability of at least one of the tissue property parameters is high.

Furthermore, according to the first embodiment, the evaluating function 455 evaluates an index value based on multiple tissue property parameters based on the reliability index. Therefore, the ultrasound diagnostic apparatus 10 according to the first embodiment can perform analysis on an index value with a high reliability index, and enables to perform analysis of the index value appropriately.

Moreover, according to the first embodiment, the control function 451 displays the index value based on multiple tissue property parameters and the reliability index on the display 2. Furthermore, the control function 451 displays the index value based on multiple tissue property parameters only for an area in which a reliability index is equal to or higher than a threshold in a tissue of a subject. Moreover, the control function 451 displays the two-dimensional color map indicating the reliability index for a region of interest in a tissue of the subject. Therefore, the ultrasound diagnostic apparatus 10 according to the first embodiment enables to perform appropriate analysis of an index value easily.

Furthermore, according to the first embodiment, the measuring function 453 calculates an index value based on multiple tissue property parameters based on the multiple tissue property parameters and a regression model or a machine learning model based on a statistical analysis. Therefore, the ultrasound diagnostic apparatus 10 according to the first embodiment enables to acquire the index value based on multiple tissue property parameters easily.

Moreover, according to the first embodiment, the calculating function 454 calculates a reliability index represented by a numerical value, a binary indicator, or a probability. Therefore, the ultrasound diagnostic apparatus 10 according to the first embodiment provide an easily understandable reliability index.

Furthermore, according to the first embodiment, the measuring function 453 calculates at least one of a mean value, a median value, and a standard deviation for a specified area for an index value based on multiple tissue property parameters. Therefore, the ultrasound diagnostic apparatus 10 according to the first embodiment can provide an analysis value of the index value.

Moreover, according to the first embodiment, the regression model is a model calculated by logistic regression. Furthermore, the machine learning model is a model acquired by support vector machine or a random forest. Therefore, the ultrasound diagnostic apparatus 10 according to the first embodiment can perform calculation of the index value appropriately.

### OTHER EMBODIMENTS

In the embodiment described above, an example in which an index value calculated from multiple tissue property parameters (SWE, SWD, ATI) is evaluated based on a reliability index has been explained. However, embodiments are not limited thereto, and a case in which an index value based on a single tissue property parameter is evaluated based on a reliability index may be applied. For example, a case in which an elasticity modulus measured by SWE is evaluated based on the reliability index described above (reliability index based on respective reliabilities of multiple tissue property parameters) may be applied. In such a case, the control function 451 may control to display only an elasticity modulus of a position at which the reliability index exceeds a threshold in a region of interest.

A term “processor” used in the above explanation signifies a central processing unit (CPU), a graphical processing unit (GPU), or a circuit, such as an application specific integrated circuit (ASIC), a programmable logic device (for example, simple programmable logic device (SPLD), complex programmable logic device (CPLD)), and a field programmable gate array (FPGA). The processor implements a function by reading and executing a program stored in a memory. Instead of storing a program in the memory, it may be configured to directly install a program in a circuit of the processor. In this case, the processor reads and executes the program installed in the circuit, to implement the function. The respective processors of the present embodiment are not limited to be configured as a single circuit for each processor, but may be configured by combining plural independent circuits as one processor, to implement its function.

The respective components of the respective devices illustrated in the explanation of the embodiment described above are of functional concept, and it is not necessarily required to be configured physically as illustrated. That is, specific forms of distribution and integration of the respective devices are not limited to the ones illustrated, and all or some thereof can be configured to be distributed or integrated functionally or physically in arbitrary units according to various kinds of loads, usage conditions, and the like. Furthermore, as for the respective processing functions performed by the respective devices, all or an arbitrary part thereof can be implemented by a CPU and a computer program that is analyzed and executed by the CPU, or can be implemented as hardware by wired logic.

Moreover, a method explained in the embodiment described above can be implemented by executing a program that has been prepared in advance by a computer such as a personal computer and a workstation. This program can be distributed through a network such as the Internet. Furthermore, this program can be recorded on a non-transitory computer-readable recording medium, such as a hard disk, a flexible disk (FD), a compact-disk read-only memory (CD-ROM), a magneto optical disk (MO), a digital versatile disk (DVD), and a flash memory including a universal serial bus (USB) memory and an SD card memory, and can be executed by being read by a computer from the non-transitory recording medium.

As explained above, according to the embodiments, appropriate analysis of an index value is enabled.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising:
processing circuitry configured to
measure a plurality of tissue property parameters based on a reflected wave signal received from a subject; and
calculate a reliability index of an index value based on the plurality of tissue property parameters, based on a reliability of each of the plurality of tissue property parameters.

2. The ultrasound diagnostic apparatus according to claim 1, wherein
the plurality of tissue property parameters include at least two of elasticity of a tissue, viscosity of a tissue, and attenuation of an ultrasonic wave.

3. The ultrasound diagnostic apparatus according to claim 2, wherein
the processing circuitry is configured to acquire a reliability regarding the elasticity and the viscosity of a tissue based on at least one of an amplitude of a shear wave, a signal-to-noise (S/N) ratio, propagation accuracy of the shear wave, and a standard deviation of numerical value.

4. The ultrasound diagnostic apparatus according to claim 2, wherein
the processing circuitry is configured to acquire a reliability regarding the attenuation of an ultrasonic wave based on at least one of accuracy of linear approximation of the reflected wave signal, multiple signals, and a structure.

5. The ultrasound diagnostic apparatus according to claim 1, wherein
the processing circuitry is configured to calculate the reliability index within an area in which each of reliabilities of the plurality of tissue property parameters is equal to or higher than a threshold, in a tissue of the subject.

6. The ultrasound diagnostic apparatus according to claim 1, wherein
the processing circuitry is configured to calculate the reliability index within an area in which at least one of reliabilities of the plurality of tissue property parameters is equal to or higher than a threshold, in a tissue of the subject.

7. The ultrasound diagnostic apparatus according to claim 1, wherein
the processing circuitry is further configured to evaluate an index value based on the plurality of tissue property parameters, based on the reliability index.

8. The ultrasound diagnostic apparatus according to claim 1, wherein
the processing circuitry is further configured to cause a display to display an index value based on the plurality of tissue property parameters and a reliability index.

9. The ultrasound diagnostic apparatus according to claim 8, wherein
the processing circuitry is configured to cause the display to display the index value based on the plurality of tissue property parameters only for an area in which the reliability index is equal to or higher than a threshold in a tissue of the subject.

10. The ultrasound diagnostic apparatus according to claim 8, wherein
the processing circuitry is configured to cause the display to display a two-dimensional color map indicating the reliability index for a region of interest in a tissue of the subject.

11. The ultrasound diagnostic apparatus according to claim 1, wherein
the processing circuitry is configured to calculate the index value based on the plurality of tissue property parameters, based on the plurality of tissue property parameters, and any one of a regression model and a machine learning model based on statistical analysis.

12. The ultrasound diagnostic apparatus according to claim 1, wherein
the processing circuitry is configured to calculate the reliability index represented by any one of a numerical value, a binary indicator, and a probability.

13. The ultrasound diagnostic apparatus according to claim 12, wherein
the processing circuitry is configured to calculate at least one of a mean value, a median value, and a standard deviation in a specified area, for the index value based on the plurality of tissue property parameters.

14. The ultrasound diagnostic apparatus according to claim 11, wherein
the regression model is a model calculated by logistic regression.

15. The ultrasound diagnostic apparatus according to claim 11, wherein
the machine learning model is a model acquired by any one of support vector machine and a random forest.

16. A method composing:
measuring a plurality of tissue property parameters based on a reflected wave signal received from a subject; and
calculating a reliability index of an index value based on the plurality of tissue property parameters, based on each of reliabilities of the plurality of tissue property parameters.

17. A storage medium that non-transitorily stores a program that causes a computer to execute:
measuring a plurality of tissue property parameters based on a reflected wave signal received from a subject; and
calculating a reliability index of an index value based on the plurality of tissue property parameters, based on each of reliabilities of the plurality of tissue property parameters.
